(19) United States
(12) Patent Application Publication
(10) Pub. No.: US 2023/0122352 A1
(43) Pub. Date: Apr. 20, 2023

(54) METHOD OF TREATING ORAL PAIN WITH AN EFFERVESCING COMPOSITION

(71) Applicants: Neal James Nesbitt, Athens, OH (US); Curtiss E. Jackson, Honolulu, HI (US)

(72) Inventors: Neal James Nesbitt, Athens, OH (US); Curtiss E. Jackson, Honolulu, HI (US)

(21) Appl. No.: 17/451,241

(22) Filed: Oct. 18, 2021

(51) Int. Cl. A61K 9/00; A61K 31/194; A61K 33/10; A61K 45/06

(52) U.S. Cl. CPC A61K 9/0056; A61K 31/194; A61K 33/10; A61K 45/06

(57) ABSTRACT

A method of treating oral pain includes administering an effervescing composition into a user's mouth. The user retains the composition with the mouth closed until the effervescing composition fully dissolves, maintaining greater than ambient pressure in the mouth and releasing only excess gas through the nose.

### BACKGROUND OF THE INVENTION

The present invention relates to treatment of oral pain and, more particularly, to a method of treating oral pain with an effervescing composition.

To treat toothache and oral pain, currently available remedies use oral anesthetics or systemic pain medicine which have side effects. These remedies are not specific to the cause of the pain.

As can be seen, there is a need for a more direct remedy treating the source of the pain without side effects.

### SUMMARY OF THE INVENTION

In one aspect of the present invention, a method of treating oral pain and/or tooth pain is provided, comprising: administering an effervescing composition into a user's mouth; and retaining the effervescing composition in the user's mouth with the user's mouth closed while the effervescing composition dissolves, maintaining a gas pressure greater than ambient, releasing only excess gas through a user's nose.

These and other features, aspects and advantages of the present invention will become better understood with reference to the following drawings, description, and claims.\

### BRIEF DESCRIPTION OF THE DRAWINGS

FIG. 1 is a cross-sectional view of a tooth employing a method of treating tooth pain with an effervescent composition according to an embodiment of the present invention; and

FIG. 2 is a block diagram of the method of FIG. 1.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a method of treating oral pain comprising applying an effervescing composition and retaining the effervescing composition in a user's mouth while it dissolves.

Effervescing compositions comprising anhydrous citric acid and sodium bicarbonate relieve the pain from toothache by direct application. For example, effervescing compositions such as Alka-Seltzer® antacid and Bromo Seltzer® antacid may be used. The effervescing compositions may be provided in the form of tablets or crystals.

In some embodiments, the effervescing composition may further include an analgesic such as aspirin and/or acetaminophen.

Applicants have discovered that persons with toothache or oral pain may get full temporary relief by placing an effervescing composition in the mouth as the effervescing composition dissolves. Without being bound by theory, the effervescing compositions are believed to work by holding most of the gas generated in the mouth. The gas generated forces carbon dioxide and bicarbonate, as well as any aspirin or acetaminophen present, down below the gum line around the tooth directly to the nerves transmitting pain, thereby relieving the pain.

A method of treating tooth pain according to an embodiment of the present invention may include the following steps. The user may place an effervescing composition in the form of crushed tablets or crystals in the mouth and let it dissolve, maintaining as much pressure as possible. For example, the gas pressure may be greater than ambient pressure, greater than ambient pressure plus 10 kPa, greater than ambient pressure plus 20 kPa, greater than ambient pressure plus 30 kPa, greater than ambient pressure plus 40 kPa, greater than ambient pressure plus 50 kPa, greater than ambient pressure plus 60 kPa, greater than ambient pressure plus 70 kPa, greater than ambient pressure plus 80 kPa, greater than ambient pressure plus 90 kPa, and greater than twice ambient pressure. Without being bound by theory, the maximum gas pressure tolerated by the human mouth is believed to be greater than twice ambient pressure. The user may allow the excess gas to escape through the nose. When the effervescence is complete, the user preferably swallows the dissolved effervescing composition and saliva and drink an amount of water, e.g., about eight ounces. The user may repeat the method after about twelve hours. The amounts and times are not particularly limited.

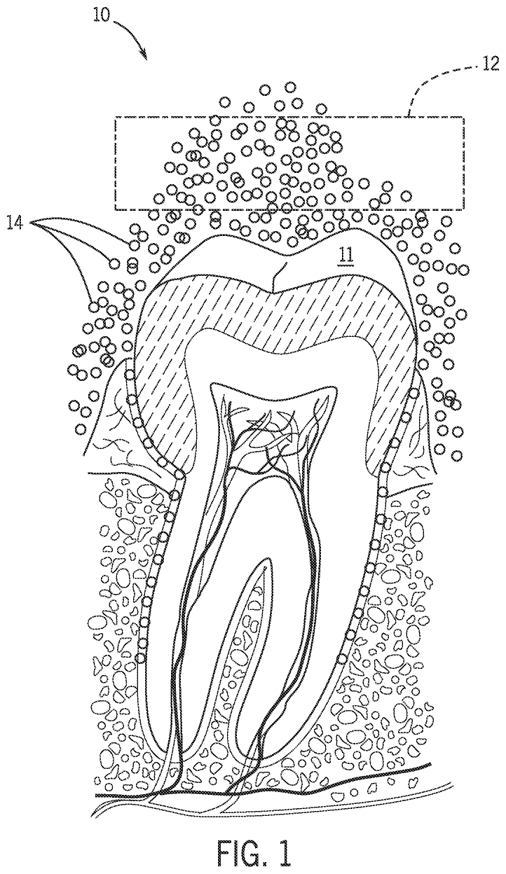

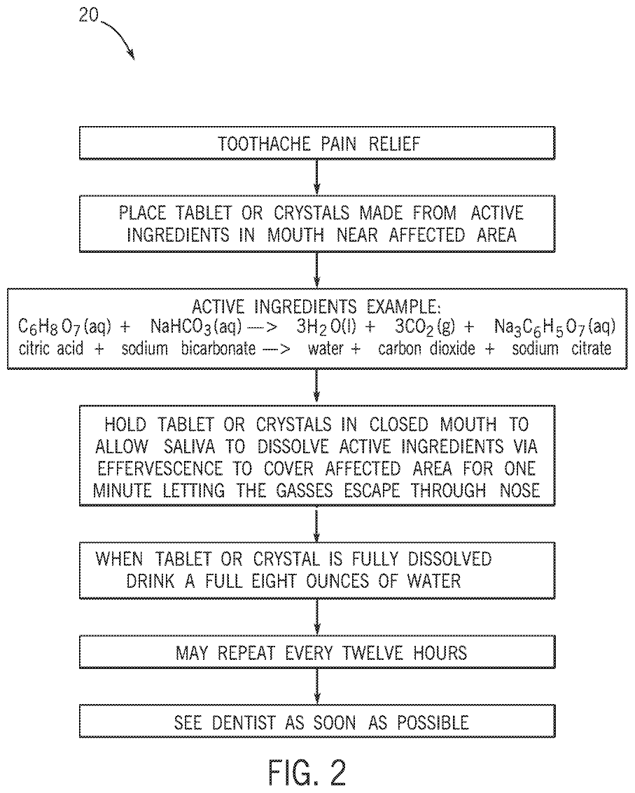

Referring to FIGS. 1 and 2, FIG. 1 is a schematic showing process of treating oral pain according to an embodiment of the present invention. An effervescent tablet or crystal 12 is placed on a tooth 11 in an oral cavity 10. As the tablet or crystal 12 dissolves, it emits effervescence 14, which works its way down the side of the tooth towards the root. Any analgesic contained within the composition may be carried with the effervescence 14. FIG. 2 provides a flowchart 20 of a method according to an embodiment of the present invention.

It should be understood, of course, that the foregoing relates to exemplary embodiments of the invention and that modifications may be made without departing from the spirit and scope of the invention as set forth in the following claims.

What is claimed is:

1. A method of treating oral pain comprising:
a) administering an effervescing composition into a user's mouth; and
b) retaining the effervescing composition in the user's mouth with the user's mouth closed while the effervescing composition dissolves, maintaining a gas pressure greater than ambient pressure, releasing only excess gas through a user's nose, until the effervescing composition is fully dissolved.

2. The method of claim 1, wherein the effervescing composition comprises anhydrous citric acid and sodium bicarbonate in a form selected from the group consisting of crushed tablets and crystals.

3. The method of claim 1, wherein the effervescing composition further comprises an analgesic.

4. The method of claim 1, wherein the gas pressure is maintained at greater than 10 kPa above ambient pressure.

5. The method of claim 1, wherein the gas pressure is maintained at greater than 20 kPa above ambient pressure.

6. The method of claim 1, wherein the gas pressure is maintained at greater than 30 kPa above ambient pressure.

7. The method of claim 1, wherein the gas pressure is maintained at greater than 40 kPa above ambient pressure.

8. The method of claim 1, wherein the gas pressure is maintained at greater twice ambient pressure.

9. The method of claim 1, further comprising:
a. swallowing the effervescing composition after the step of retaining; and
b. drinking about 8 ounces of water.

10. The method of claim 9, further comprising:
repeating the administering, retaining, swallowing, and drinking after about 12 hours.
